(19) United States
(12) Patent Application Publication
(10) Pub. No.: US 2022/0119856 A1
(43) Pub. Date: Apr. 21, 2022

(54) STALL SIDE METHOD FOR THE DETECTION OF BACTERIA IN DAIRY CATTLE

(71) Applicant: TRANSFORMATIVE TECHNOLOGIES, Baltimore, MD (US)

(72) Inventors: Lawrence SILVER, Westbury, NY (US); Lawrence LOOMIS, Columbia, MD (US); David DONOVAN, Baltimore, MD (US)

(73) Assignee: TRANSFORMATIVE TECHNOLOGIES, Baltimore, MD (US)

(21) Appl. No.: 17/427,587

(22) PCT Filed: Jan. 30, 2020
(86) PCT No.: PCT/US2020/015862

§ 371 Date: Jul. 30, 2021

Related U.S. Application Data
(60) Provisional application No. 62/799,058, filed on Jan. 31, 2019.

(51) Int. Cl. C12Q 1/14; C12Q 1/06

(52) U.S. Cl. CPC C12Q 1/14; C12Q 1/06

(57) ABSTRACT

The present invention relates to several methods to detect gram positive mastitis pathogens in a small sample of bovine milk by luminescence using a combination of specific reagents giving a “cow side” “in-stall” indication of the presence or absence of gram positive mastitis pathogens within a short period of time.

### CROSS REFERENCE TO RELATED APPLICATIONS

The present application is a U.S. National Phase Application under 35 U.S.C. § 371 of International Patent Application No. PCT/US2020/015862 filed Jan. 30, 2020, which claims priority of U.S. Provisional Patent Application No. 62/799,058 filed Jan. 31, 2019. The entire contents of which are hereby incorporated by reference.

### GOVERNMENT LICENSE RIGHTS

This invention was made with government support under 58-3K95-4-1707-M awarded by the United States Department of Agriculture. The government has certain rights in the invention.

### FIELD OF THE INVENTION

The present invention relates to several methods to detect gram positive mastitis pathogens in a small sample of bovine milk by luminescence using a combination of specific reagents giving a “cow side” “in-stall” indication of the presence or absence of gram positive mastitis pathogens within a short period of time.

For the purposes of the present invention, all references as cited herein are incorporated by reference in their entireties.

### BACKGROUND

Dairy cattle mastitis is the most costly disease to the dairy industry costing more than $2 billion annually in losses due to cost of veterinary visits, antibiotic treatment, reductions in milk quality and quantity and in the most severe cases, animal culling. It is also responsible for the largest amount of antibiotic use in the dairy industry. Infections are usually mono-specie. There are currently no tools to allow the dairy farmer to quickly determine (e.g. by visual inspection or rapid diagnostic) if a mastitic cow is infected with either Gram negative or Gram positive pathogens. Gram status determinations could greatly reduce the cost and improve the efficacy of mastitis treatment because Gram positive pathogens are generally responsive (susceptible) to antibiotic treatment while Gram negative organisms are generally refractory to these treatments. Currently, Gram status determinations require differential bacterial plate culturing [under sterile conditions] that with transport, usually take 24-36 hours for reliable results. This delay is detrimental to the dairy farmer at many levels including milk yields, milk quality, animal health, and increased risk of contagious pathogen spreading through the herd. In order to keep veterinary costs down, decisions on whether or not to administer antibiotics to combat mastitis are often left in the hands of the milking parlor attendant. A rapid diagnostic tool that would allow the farmer or milking parlor attendant to diagnose Gram positive mastitis pathogens in dairy cows within the ˜10 minutes required to milk the cow, would potentially be of huge benefit to the dairy industry.

The majority (>95%) of the Gram positive mastitis causing pathogens in the US are multiple species: Streptococcus uberis and staphylococci (Staphylococcus aureus and Coagulase negative staphylococci). Thus as opposed to identifying all putative Gram Positive organisms in milk, it is most relevant if we can identify these streptococci and staphylococci in milk. Gram status determinations could greatly reduce the cost and improve the efficacy of mastitis treatment because Gram positive pathogens are generally responsive (susceptible) to antibiotic treatment. While antibiotics are not recommended for Gram negative pathogens, since Gram negative infectious generally cure themselves such that treatment is not warranted (Van Eenennaam et al., 1995; Wilson et al., 1999). Using this logic, Roberson (Roberson, 2003), estimated that antibiotics would not be warranted in 50-80% of mastitis cases. There are no tools to allow the dairy farmer to quickly determine (e.g. by visual inspection or rapid diagnostic) if a mastitic cow is infected with either Gram negative or Gram positive pathogen, which results in over treatment, and increased use of antibiotics. Another significant cost is the use of antibiotics at dry-off. It is customary to treat every quarter of every cow at dry-off as a preventive measure. If an inexpensive Gram positive diagnostic test existed, the use antibiotics on the dairy farm could be greatly reduced.

Currently, Gram status determinations require differential bacterial plate culturing [under sterile conditions] or PCR diagnostics at an off site facility (shipping usually take 24-36 hours) for reliable results. We reason that a rapid diagnostic test identifying the key Gram positive mastitis pathogens could be a significant savings to the dairy farmer. Rapid diagnosis and quarantine/treatment would prevent the spread of these contagious pathogens throughout the herd, and could readily save the costs of the current treatments that are wasted on Gram negative pathogens as well as reduce overall antibiotic use. Due to concerns regarding resistance transfer from farm to clinic, reduction in broad range antibiotic use is also a concept supported by the Transatlantic Taskforce on Antimicrobial Resistance, including USDA, CDC, NIH, EU regulatory agencies (www.cdc.gov) and most recently FDA with proposed restrictions on antibiotic use in animal feeds. We predict that our bioluminescence diagnostic will cost approximately $8-10 per test making this a highly competitive and commercializable assay.

There are numerous citations on the web indicating that if rapid diagnostics were available to identify mastitis caused by Gram positive pathogens; these would be the cases that would be targeted with antibiotics. A quote from the 47th annual meeting in 2008 of the US National Mastitis Counsel indicates that:

“ . . . The vast majority of subclinical intramammary infections are caused by Gram positive bacteria . . . in a three-year U.S./Canadian study . . . researchers evaluated 4,044 quarters from 1,028 fresh cows in 11 distinct herds. Of the intramammary infections (IMI) detected, a striking 91% were shown to be caused by Gram positive pathogens. Furthermore, of the relatively small number of infections caused by Gram negative bacteria, most self-cure without treatment” (Dairybusiness.com). Another web site indicates: “ . . . cows with streptococcal or staphylococcal mastitis are more likely than cows with coliform mastitis to respond to antibiotic therapy” 1998 (www.livestocktrail.illinois.edu). Also, according to a study carried out in Israel: Out of 6878 cases of mastitis tested 37% was caused by Gram positive while only 2.6% was due to Gram negative (www.halavi.org.il). The majority of bovine mastitis was previously caused by infectious pathogens (e.g., Staph. aureus, Streptococcus agalactiae; (Hillerton and Berry, 2005)). However, improvements in herd management, e.g. antibiotic intervention, has reduced the frequency of infectious bovine mastitis (Bradley, 2002; Hillerton and Berry, 2005). Environmental mastitis (primarily Streptococcus uberis and E. coli) has been increasing. Also, coagulase negative staphylococci (CoNS) seem to be an emerging concern (Pyorala and Taponen, 2009).

The pathogens found most common in milk varies with both geographic region and year of testing e.g. in Europe at dry off the primary pathogens were Corynebacterium spp. (37%), CoNS (19%), S. uberis (2%) and S. aureus (2%) (Bradley et al., 2015); in two studies in Thailand, the major pathogens are Streptococcus (spp.) (16.4%; --), S. uberis (9.4%; 13.8%), S. agalactiae (7.1%; --), S. aureus (2.9%; 5.4%), Corynebacterium (--; 4.5%), S. dysgalactiae (4.0%; --) and CoNS (--; 9.9%) (Leelahapongsathon et al., 2014; Suriyasathaporn et al., 2012), respectively. The most recent testing for US is from 1997 (Wilson et al., 1997) with S. aureus and streptococcal pathogens representing >50% of the mastitis pathogens.

There are currently two broadly available mastitis tests for monitoring milk quality. One assay, called the Somatic Cell Count (SCC) determines the level of somatic cells in the milk. The weaknesses of the SCC assay include inaccuracy, since it is negatively influenced by the presence of pathogens (the primary cause of mastitis) in the milk; insensitivity as it only provides a threshold value of the levels of somatic cells; and failure to provide early-diagnostic information, because the results are provided to the farm up to a month after the initial acquisition of milk samples. Moreover, an individual infected animal is not identified because SCC levels are typically measured in the bulk milk reservoir, which can contain milk from 50-100 cows. Therefore, this compromises the quality of milk in the bulk reservoir and delays detection of the affected animal. Moreover, advanced mastitis necessitates more aggressive treatment, prolonged withdrawal of the animal from the milk line, and a higher probability of generating a chronically affected individual, all of which represent significant economic liabilities to the farm. The SCC assay is hindered by the expense and delay of testing samples at a remote laboratory. Significantly, the delay prevents the implementation of prompt remedial action. Generally, the farmer/field agent recognizes symptoms in an affected animal and removes it from the milk line. However, this represents an action after the infection has occurred.

The second assay is termed the California Mastitis Test (CMT). In this method, milk from each quadrant of the udder is deposited into each of four shallow receptacles, to which a proprietary solution is added. Gentle mixing results in clumping of mastitis-positive samples. This is an imprecise assay that does not give any quantitative measurement of the level of infection. Moreover, the assay is quite insensitive, and does not detect lower levels of persistent mastitis.

Clinical mastitis causes greater than $2 billion in directly attributable losses for the dairy industry. However, this is an underestimate, because the financial loss caused by low quality milk and poor yield from sub-clinical cows, treatment of affected animals, withdrawal from the milk line, and occasional culling of ill animals is not estimated. Accordingly, there is a need in the art for rapid, reliable, inexpensive and accurate tests for detecting mastitis.

### SUMMARY OF THE INVENTION

The present invention features methods and kits for detecting and monitoring mastitis. In one embodiment, a method for determining amounts of bacteria in bovine milk comprising the steps of:
i) Filtration of the bovine milk to produce a filtrate
ii) Treatment of the filtrate with a non ionic surfactant which lyses non microbial cells (somatic cells) to produce a first solution
iii) Treatment of the first solution with an ATP eliminating enzyme to produce a second solution
iv) Treatment of the second solution with an ATP eliminating enzyme inhibitor to give a third solution
v) Treatment of the third solution with a microbial lysing agent to give a fourth solution
vi) Treatment of the fourth solution with a Luciferin/Luciferase reagent to give a fifth solution and
vii) Quantitation of bacteria in the fifth solution by luminescence;
   is described.

In a further embodiment the bovine milk is obtained from a surface using a cloth, gauze, swab, wipe, non-woven fiber or sponge. In a further embodiment the non-ionic surfactant is chosen from the group consisting of Neonol AF9-10 (Nonoxynol-9), saponin, amphipathic glycosides Triton X-100 and Lubrol, preferably Neonol AF9-10.

In a further embodiment the ATP eliminating enzyme comprises at least one member selected from the group consisting of apyrase, alkaline phosphatase, acidic phosphatase, hexokinase, adenosine triphosphatase, and adenosine phosphate deaminase, preferably apyrase.

In another embodiment the ATP eliminating enzyme inhibitor is an ionic surfactant selected from the group consisting of anionic surfactants, cationic surfactants and zwitterion surfactants.

In another embodiment the anionic surfactant is selected from the group consisting of alkyl sulfates, alkyl ether sulfates, docusates, sulfonate fluorosurfactants, alkyl benzene sulfonates, alkyl aryl ether phosphates, alkyl ether phosphates, alkyl carboxylates, and carboxylate fluorosurfactants, more preferably selected from the group consisting of ammonium lauryl sulfate, sodium dodecyl sulfate (SDS), sodium deoxycholate, sodium-n-dodecylbenzenesulfonate, sodium lauryl ether sulfate (SLES), sodium myreth sulfate, dioctyl sodium sulfosuccinate, perfluorooctanesulfonate (PFOS), perfluorobutanesulfonate, sodium stearate, sodium lauroyl sarcosinate, perfluorononanoate, and perfluorooctanate (PFOA or PFO).

In another embodiment the cationic surfactant is selected from the group consisting of cetyl trimethylammonium bromide (CTAB), cetyl trimethylammonium chloride (CTAC), cetylpyridinium chloride (CPC), Polyethoxylated tallow amine (POEA), benzalkonium chloride (BAC), benzthonium chloride (BZT), 5-bromo-5-nitro-1,3-dioxane, dimethyldioctadecylammonium chloride, laureltrimethylammonium bromide (DTAB), benzyldimethyldodecylammonium bromide (BDDABr), dioctadecyldimethylammonium bromide (DODAB).

Preferably the ionic surfactant is selected from DTAB, CTAB and BDDABr.

In another embodiment the zwitterion surfactant is sulfobetaine-3-10.

In another embodiment the ATP eliminating enzyme inhibitor is selected from the group consisting of vanadates and hydroxyapatites and their derivatives.

In another embodiment the microbial lysing agent is a bacteriophage lytic enzyme (endolysin) or modified lytic enzyme (genetic or chimeric). Preferably the bacteriophage lytic endolysin is selected from lysostaphin, LysK, lambdaSa2, OSH3b, and KSN383, lysA, lysA2, LysgaY, truncated lambda Sa2 and plyC.

In another embodiment the Luciferin/Luciferase reagent is chosen from the group consisting of Hygiena ATP Biomass Kit #CCK4, Promega Bright Glo system and any formulations which contain naturally occurring or genetically recombinant Luciferase.

In another embodiment the quantization of bacteria is performed on a liquid or solid state substrate, preferably on a solid state substrate.

In another embodiment the solid-state substrate is selected from polyvinyl alcohol, Porex membrane, Whatman paper membranes, Ahlstrom membranes, Nitrocellulose membranes, and Whatman Nytran membranes, Nylon membranes and paper.

In another embodiment the bacteria is gram positive bacteria.

In another embodiment the gram positive bacteria is selected from Staphylococcus spp., Streptococcus spp., Propionibacterium spp., Enterococcus spp., Bacillus spp., Corynebacterium spp., Nocardia spp., Clostridium spp., Actinobacteria spp., Lactococcus spp. and Listeria spp.

In another embodiment the bacteria is selected from the group consisting of streptococcus agalactiae, streptococcus spp., staphylococcus aureus and staphylococcus spp.

In a second embodiment a method for determining amounts of bacteria in bovine milk comprising the steps of:
i) Contacting the bovine milk with a material which selectively attracts bacteria
ii) Separating the bacteria from the material and placing the bacteria into solution
iii) Treatment of the bacterial solution with a bacterial releasing agent capable of lysing bacterial cells to give a released ATP second solution
iv) Treatment of the second solution with a Luciferin/Luciferase reagent to give a third solution and
v) Quantitation of bacteria in the third solution by luminescence;
   is described.

In another embodiment the material is selected from the group consisting of antibody coated surfaces, lectin coated surfaces, lytic enzyme binding domains coated surfaces, glass wool membranes and treated glass surfaces or any charged or uncharged surface.

In another embodiment the bovine milk is obtained from a surface using a cloth, gauze, swab, wipe, non woven fiber or sponge.

In another embodiment the bacterial releasing agent is a bacteriophage lytic enzyme (endolysin) or modified lytic enzyme (genetic or chimeric), quaternary amines, ionic and or non-ionic surfactants.

In another embodiment the ionic surfactant is selected from the group consisting of anionic surfactants cationic surfactants and zwitterion surfactants.

In another embodiment the anionic surfactant is selected from the group consisting of alkyl sulfates, alkyl ether sulfates, docusates, sulfonate fluorosurfactants, alkyl benzene sulfonates, alkyl aryl ether phosphates, alkyl ether phosphates, alkyl carboxylates, and carboxylate fluorosurfactants, more preferably selected from the group consisting of ammonium lauryl sulfate, sodium dodecyl sulfate (SDS), sodium deoxycholate, sodium-n-dodecylbenzenesulfonate, sodium lauryl ether sulfate (SLES), sodium myreth sulfate, dioctyl sodium sulfosuccinate, perfluorooctanesulfonate (PFOS), perfluorobutanesulfonate, sodium stearate, sodium lauroyl sarcosinate, perfluorononanoate, and perfluorooctanate (PFOA or PFO).

In another embodiment the cationic surfactant is selected from the group consisting of cetyl trimethylammonium bromide (CTAB), cetyl trimethylammonium chloride (CTAC), cetylpyridinium chloride (CPC), Polyethoxylated tallow amine (POEA), benzalkonium chloride (BAC), benzthonium chloride (BZT), 5-bromo-5-nitro-1,3-dioxane, dimethyldioctadecylammonium chloride, laureltrimethylammonium bromide (DTAB), benzyldimethyldodecylammonium bromide (BDDABr), dioctadecyldimethylammonium bromide (DODAB).

In another embodiment the ionic surfactant is selected from DTAB, CTAB and BDDABr.

In another embodiment the zwitterion surfactant is sulfobetaine-3-10.

In another embodiment the bacteriophage lytic endolysin is selected from lysostaphin, LysK, lambdaSa2, OSH3b, and KSN383, lysA, lysA2, LysgaY, truncated lambda Sa2 and plyC.

In another embodiment the Luciferin/Luciferase reagent is chosen from the group consisting of Hygiena ATP Biomass Kit #CCK4, Promega Bright Glo system and any formulations which contain naturally occurring or genetically recombinant Luciferase.

In another embodiment the bacteria is gram positive bacteria.

In another embodiment the gram positive bacteria is selected from Staphylococcus spp., Streptococcus spp., Propionibacterium spp., Enterococcus spp., Bacillus spp., Corynebacterium spp., Nocardia spp., Clostridium spp., Actinobacteria spp., Lactococcus spp. and Listeria spp.

In another embodiment the bacteria is selected from the group consisting of streptococcus agalactiae, streptococcus spp., staphylococcus aureus and staphylococcus spp.

In a third embodiment a method for determining amounts of bacteria in bovine milk comprising the steps of:
i) Placing the bovine milk in a first container containing a non ionic surfactant which lyses non microbial (somatic cells) and an ATP eliminating enzyme to produce a first solution;
ii) Transferring the first solution to a second container containing an ATP eliminating enzyme inhibitor, a microbial lysing agent and a Luciferin/Luciferase reagent to give a second solution and
iii) Quantitation of bacteria in the second solution by luminescence
   is described.

In another embodiment the bovine milk is obtained from a surface using a cloth, gauze, swab, wipe, non woven fiber or sponge.

In another embodiment the non-ionic surfactant is chosen from the group consisting of Neonol AF9-10 (Nonoxynol-9), saponin, amphipathic glycosides Triton X-100 and Lubrol, preferably the non-ionic surfactant is Neonol AF9-10.

In another embodiment the ATP eliminating enzyme comprises at least one member selected from the group consisting of apyrase, alkaline phosphatase, acidic phosphatase, hexokinase, adenosine triphosphatase, and adenosine phosphate deaminase, preferably the ATP eliminating enzyme is Apyrase.

In another embodiment the ATP eliminating enzyme inhibitor is an ionic surfactant.

In another embodiment the ionic surfactant is selected from the group consisting of anionic surfactants cationic surfactants and zwitterion surfactants.

In another embodiment the anionic surfactant is selected from the group consisting of alkyl sulfates, alkyl ether sulfates, docusates, sulfonate fluorosurfactants, alkyl benzene sulfonates, alkyl aryl ether phosphates, alkyl ether phosphates, alkyl carboxylates, and carboxylate fluorosurfactants, more preferably selected from the group consisting of ammonium lauryl sulfate, sodium dodecyl sulfate (SDS), sodium deoxycholate, sodium-n-dodecylbenzenesulfonate, sodium lauryl ether sulfate (SLES), sodium myreth sulfate, dioctyl sodium sulfosuccinate, perfluorooctanesulfonate (PFOS), perfluorobutanesulfonate, sodium stearate, sodium lauroyl sarcosinate, perfluorononanoate, and perfluorooctanate (PFOA or PFO).

In another embodiment the cationic surfactant is selected from the group consisting of cetyl trimethylammonium bromide (CTAB), cetyl trimethylammonium chloride (CTAC), cetylpyridinium chloride (CPC), Polyethoxylated tallow amine (POEA), benzalkonium chloride (BAC), benzthonium chloride (BZT), 5-bromo-5-nitro-1,3-dioxane, dimethyldioctadecylammonium chloride, laureltrimethylammonium bromide (DTAB), benzyldimethyldodecylammonium bromide (BDDABr), dioctadecyldimethylammonium bromide (DODAB).

In another embodiment the ionic surfactant is selected from DTAB, CTAB and BDDABr.

In another embodiment the zwiterionic surfactant is sulfobetaine-3-10.

In another embodiment the ATP eliminating enzyme inhibitor is selected from the group consisting of vanadates and hydroxyapatites and their derivatives.

In another embodiment the microbial lysing agent is a bacteriophage lytic enzyme (endolysin) or modified lytic enzyme (genetic or chimeric).

In another embodiment the bacteriophage lytic endolysin is selected from lysostaphin, LysK, lambdaSa2, OSH3b, and KSN383, lysA, lysA2, LysgaY, truncated lambda Sa2 and plyC.

In another embodiment the Luciferin/Luciferase reagent is chosen from the group consisting of Hygiena ATP Biomass Kit #CCK4, Promega Bright Glo system and any formulations which contain naturally occurring or genetically recombinant Luciferase.

In another embodiment the bacteria is gram positive bacteria.

In another embodiment the gram positive bacteria is selected from Staphylococcus spp., Streptococcus spp., Propionibacterium spp., Enterococcus spp., Bacillus spp., Corynebacterium spp., Nocardia spp., Clostridium spp., Actinobacteria spp., Lactococcus spp and Listeria spp.

In another embodiment the bacteria is selected from the group consisting of streptococcus agalactiae, streptococcus spp., staphylococcus aureus and staphylococcus spp.

### DETAILED DESCRIPTION

### EXAMPLES

The following examples are intended to illustrate the present invention without limitations.

### Example 1

### Determination of Usefulness of Luciferin/Luciferase Reagent in Measuring ATP Concentrations in Raw Bovine Milk Sample

Raw Bovine milk samples are known to have endogenous ATP which potentially could interfere with a bioluminescent assay. Raw Bovine milk samples were prepared containing various amounts of ATP standard from Sigma Chemical (#A2383.) to produce raw Bovine milk samples with concentrations of 10⁻⁶M to 10⁻¹⁰M. In each test, 50 uL of Promega luciferin-luciferase reagent (containing 25 mM HEPES buffer (pH 7.5), 40 pg luciferase, 100 pM luciferin, and 10 mM MgSO4). were added to 50 uL of Bovine milk sample and light output was determine using Hygiena Ensure System. All measurements were performed by first obtaining signal output of raw Bovine milk sample devoid of additional ATP and final ATP readings for each sample described above were corrected for the blank. In all cases the ATP levels were detected as expected for the ATP concentrations noted above. For comparison, standard solutions of ATP prepared in buffer were tested against their blank, and results similar to the raw Bovine milk study were obtained. This indicates that raw Bovine milk samples do not appear to hinder the Luciferin-Luciferase reaction, and are useful for the determination of bacteria in raw milk.

### Example 2

### Determination of Optimum Method to Filter Bovine Milk Sample

Some assays it may be necessary to pretreat the milk sample to remove fats and other endogenous materials that may be present in raw Bovine milk. In this procedure, raw Bovine milk (with endogenous bacteria) was filtered via gravity flow for 2 min using 2 different commercially available filter papers (after evaluation of numerous filter media). The filter media of choose are: Ahlstrom 222 (A222) and Ahlstrom 142 (A142). The filter papers where supplied by Ahlstrom Corporation. Samples (100 uL) prior to and after filtering on Ahlstrom 222 (A222) and Ahlstrom 142 (A142)) were tested for ATP with 50 uL of Promega luciferin-luciferase reagent (containing 25 mM HEPES buffer (pH 7.5), 40 pg luciferase, 100 pM luciferin, and 10 mM MgSO4), as well as for colony forming units (CFUs) via serial dilution on rich media tryptic soy agar (TSA) plates. The endogenous ATP appears to have largely (70%) bound to the A222 filter, while nearly 95% of the bacteria appear to have passed through. The A142 filter does not appear useful in this process since it does not allow the bacteria to pass through. There was no testing for somatic cells in the filtrate of the A222 filter, such that the reduction in ATP after filtration might reflect the capturing of the somatic cells on the filter and the resultant loss of intracellular somatic cells stores of ATP. All determinations of ATP concentration were performed using a Hygiena Ensure luminometer.

### Example 3

### Determination of Methods to Rupture Somatic Cells Present in Raw Bovine Milk Samples

A number of surfactants (detergents) were evaluated for their ability to rupture the somatic cells present in raw Bovine milk samples. Among the reagents tested were Triton X100 (Sigma Chemical) and Neonol AF9-10 (Nonoxynol-9) (Elarum Petrochemicals). Determinations using both surfactants were performed on raw Bovine milk samples in which somatic cell counts were predetermined. It was determined that the Neonol-9-10 was superior to the Triton X100 in its ability to rupture somatic cells in under 90 seconds. The number of somatic cell ruptured was determined by quantifying the ATP released in 50 uL samples of treated raw Bovine milk (as a function of time) using 50 uL of Promega luciferin-luciferase reagent (containing 25 mM HEPES buffer (pH 7.5), 40 pg luciferase, 100 pM luciferin, and 10 mM MgSO4). Bioluminescent measurements were performed using a Hygiena Ensure luminometer and all readings were blank corrected.

### Example 4

### Determination of Method to Eliminate Endogenous ATP Present in Raw Bovine Milk Sample

To demonstrating the ability to eliminate the endogenous ATP from raw Bovine milk, we selected Apyrase, an ATPase enzyme, from Sigma Chemical (A6535, ATPase ≥200 units/mg protein). All assays were performed using 50 uL of raw Bovine milk and 50 uL of Promega luciferin-luciferase reagent (containing 25 mM HEPES buffer (pH 7.5), 40 pg luciferase, 100 pM luciferin, and 10mM MgSO4). Bioluminescent measurements were determined using the Hygiena Ensure System. The test was performed in both 100% and 50% raw Bovine milk. Results indicate that Apyrase works well in both 100% and 50% raw Bovine milk samples. At a concentration of 284 mUnits the apyrase is able to deplete the endogenous ATP in 50 μL of raw milk in less than 30 seconds. The diminution of ATP was so fast with higher concentrations of Apyrase (diluting the enzyme) resulting in the inability to take meaningful reading, since all of the ATP was gone within 10 seconds. There are numerous commercially available ATP-degrading enzymes that can be tested for this purpose, but in our system, the Apyrase enzyme appears more than sufficient.

### Example 5

### Determination of Method to Evaluate “Eliminating Reagents” for Excess Apyrase Enzyme Present in Raw Bovine Milk Sample after Treatment to Eliminate Endogenous ATP (Example #4)

It is important to eliminate any excess Apyrase that may remain in the raw Bovine milk sample after treatment with the Apyrase enzyme that was used to eliminate endogenous ATP in Example #4. We examined a number of anionic and cationic surfactants (detergents) to determine those that are most effective at inactivate Apyrase. Among the reagents evaluated were: dimethyldioctadecylammonium chloride, laureltrimethylammonium bromide (DTAB), benzyldimethyldodecylammonium bromide (BDDABr), and cetyl trimethylammonium bromide (CTAB). The experiment was performed by adding between 0.02% and 1% of the selected surfactant to 50 ul of raw Bovine milk that had treated previously been treated with 284 mU of Apyrase as detailed in example #4. The levels of ATP were confirmed by adding 50 uL of Promega luciferin-luciferase reagent (containing 25 mM HEPES buffer (pH 7.5), 40 pg luciferase, 100 pM luciferin, and 10 mM MgSO4). As expected the readings determined on the Hygiena Ensure Luminometer were too low to measure, since all of the ATP had already been eliminated by Apyrase treatment in Example #4. After the initially readings were determined as described above, 10 uL of a 10⁻⁸M solution of ATP standard (Sigma Chemical) was added to the samples above and the bioluminescent signal was determined on a Hygiena Ensure Luminometer. As expected a signal was now detected, since all of the excess Apyrase added earlier had been eliminated by the surfactants being evaluated. The best results were seen when the detergents benzyldimethyldodecylammonium bromide (BDDABr) at 0.05% concentration and, laureltrimethylammonium bromide (DTAB) at 0.5% and 1.0% concentrations were added to the samples.

### Example 6

### Demonstrate the Ability of Both Streptococcal and Staphylococcal Phage Lytic Enzyme (Endolysins or Peptidoglycan Hydrolases) to Effectively Rupture Gram Positive Bacteria in a Raw Bovine Milk Sample

As the goal of this invention is to target and detect only gram positive organisms active in mastitis such as: S. aureus, Coagulase negative staphylococci (CoNS) and S. uberi, it is important to identify lytic enzymes that can effectively rupture the cell walls of these pathogens in the presence of raw Bovine milk. Lysostaphin (Lyso) and Streptococcal phage endolysin (PlyC), as well many other phage lytic enzymes, some which were developed by Donovan, have been shown to have high activity against the major Gram positive mastitis pathogens. The action against specific gram-positive organisms of these enzymes has been verified in PBS buffered solutions, but their ability to rupture bacteria had to be verified to in raw milk. The first candidates tested in for their activity in Bovine milk were Lysostaphin (Lyso) which attacks S. aureus, and Streptococcal phage lytic enzyme (PlyC) which attacks Streptococcus uberis. In all reactions, a concentration a of 0.05% of the phage lytic enzyme was used to evaluate the time required to rupture the cell wall of gram-positive bacteria in raw Bovine milk, as well as in a Phosphate-buffered saline (PBS) solution. It was determined that that in one hour, PlyC can eradicate up to 6 logs of S. uberis in PBS buffer, and either mastitic milk or healthy milk. The degree of lysing was determined by bioluminescence produced by the ATP released from the ruptured cells as described earlier. We then lowered the bacterial load to 2 logs of S. uberis in PBS buffer, and either mastitic milk or healthy milk, and it was determined that the PlyC can eradicate this bacterial load in 3 minutes or less. Similar experiments were performed with Lysostaphin (Lyso), which attacks S. aureus, and we determined we that can eradicate up to 6 logs of S. aureus in PBS buffer, and either mastitic milk or healthy milk in under 25 minutes. We then lowered the bacterial load to 2 logs of S. aureus in PBS buffer and either mastitic milk or healthy milk, and it was determined that the Lyso can eradicate this bacterial load in 2 minutes or less.

What is claimed is:

1-22. (canceled)

23. A method for determining amounts of bacteria in bovine milk comprising the steps of:
i) Contacting the bovine milk with a material which selectively attracts bacteria
ii) Separating the bacteria from the material and placing the bacteria into solution
iii) Treatment of the bacterial solution with a bacterial releasing agent capable of lysing bacterial cells to give a released ATP second solution
iv) Treatment of the second solution with a Luciferin/Luciferase reagent to give a third solution and
v) Quantitation of bacteria in the third solution by luminescence;

24. The method according to claim 23 wherein the material is selected from the group consisting of antibody coated surfaces, lectin coated surfaces, lytic enzyme binding domains coated surfaces, glass wool membranes and treated glass surfaces or any charged or uncharged surface.

25. The method of claim 23 wherein the bovine milk is obtained from a surface using a cloth, gauze, swab, wipe, non woven fiber or sponge.

26. The method according to claim 23 wherein the bacterial releasing agent is a bacteriophage lytic enzyme (endolysin) or modified lytic enzyme (genetic or chimeric), quaternary amines, ionic and or non-ionic surfactants.

27. The method according to claim 26 wherein the ionic surfactant is selected from the group consisting of anionic surfactants cationic surfactants and zwitterion surfactants.

28. The method according to claim 27 wherein the anionic surfactant is selected from the group consisting of alkyl sulfates, alkyl ether sulfates, docusates, sulfonate fluorosurfactants, alkyl benzene sulfonates, alkyl aryl ether phosphates, alkyl ether phosphates, alkyl carboxylates, and carboxylate fluorosurfactants, more preferably selected from the group consisting of ammonium lauryl sulfate, sodium dodecyl sulfate (SDS), sodium deoxycholate, sodium-n-dodecylbenzenesulfonate, sodium lauryl ether sulfate (SLES), sodium myreth sulfate, dioctyl sodium sulfosuccinate, perfluorooctanesulfonate (PFOS), perfluorobutanesulfonate, sodium stearate, sodium lauroyl sarcosinate, perfluorononanoate, and perfluorooctanate (PFOA or PFO).

29. The method according to claim 27 wherein the cationic surfactant is selected from the group consisting of cetyl trimethylammonium bromide (CTAB), cetyl trimethylammonium chloride (CTAC), cetylpyridinium chloride (CPC), Polyethoxylated tallow amine (POEA), benzalkonium chloride (BAC), benzthonium chloride (BZT), 5-bromo-5-nitro-1,3-dioxane, dimethyldioctadecylammonium chloride, laureltrimethylammonium bromide (DTAB), benzyldimethyldodecylammonium bromide (BDDABr), dioctadecyldimethylammonium bromide (DODAB).

30. The method according to claim 27 wherein the ionic surfactant is selected from DTAB, CTAB and BDDABr.

31. The method according to claim 27 wherein the zwitterion surfactant is sulfobetaine-3-10.

32. The method according to claim 26 wherein the bacteriophage lytic endolysin is selected from lysostaphin, LysK, lambdaSa2, OSH3b, and KSN383, lysA, lysA2, LysgaY, truncated lambda Sa2 and plyC.

33. The method according to claim 23 wherein the Luciferin/Luciferase reagent is chosen from the group consisting of Hygiena ATP Biomass Kit #CCK4, Promega Bright Glo system and any formulations which contain naturally occurring or genetically recombinant Luciferase.

34. The method according to claim 23 wherein the bacteria is gram positive bacteria.

35. The method according to claim 34 wherein the gram positive bacteria is selected from Staphylococcus spp., Streptococcus spp., Propionibacterium spp., Enterococcus spp., Bacillus spp., Corynebacterium spp., Nocardia spp., Clostridium spp., Actinobacteria spp., Lactococcus spp. and Listeria spp.

36. The method according to claim 23 wherein the bacteria is selected from the group consisting of streptococcus agalactiae, streptococcus spp., staphylococcus aureus and staphylococcus spp.

37-55. (canceled)
